(19) United States
(12) Patent Application Publication
(10) Pub. No.: US 2019/0307827 A1
(43) Pub. Date: Oct. 10, 2019

(54) METHOD FOR INHIBITING EXPRESSION OF ICAM-1 GENE

(71) Applicant: TCI CO., LTD., Taipei (TW)

(72) Inventors: Yung-Hsiang LIN, Taipei (TW); Cheng-Shing LI, Taipei (TW); Kai-Wen KAN, Taipei (TW); Ciao-Ting CHEN, Taipei (TW); Fu-Chen LIU, Taipei (TW)

(21) Appl. No.: 16/103,795

(22) Filed: Aug. 14, 2018

(30) Foreign Application Priority Data
Apr. 10, 2018 (TW) 107112288

(51) Int. Cl. A61K 36/62; A23L 33/105

(52) U.S. Cl. CPC A61K 36/62; A23V 2002/00; A23L 33/105

(57) ABSTRACT

A method for inhibiting the expression of ICAM-1 gene is provided, wherein the method comprises administering to a subject in need an effective amount of an euryale seed extract. The method is especially for treating, preventing or regulating diseases or physiological functions associated with ICAM-1 gene.

### CROSS REFERENCE TO RELATED APPLICATIONS

This application claims priority to Taiwan Patent Application No. 107112288 filed on Apr. 10, 2018, in the Taiwan Intellectual Property Office, the disclosure of which is incorporated herein in their entirety by reference.

### FIELD OF THE INVENTION

The present invention relates to the uses of an euryale seed extract in inhibiting the expression of ICAM-1 gene in a subject in need, including treating, preventing or regulating diseases or physiological functions associated with ICAM-1 gene. The uses especially include treating and/or preventing cardiovascular diseases.

### BACKGROUND OF THE INVENTION

Over recent years, the incidence of cardiovascular diseases has been consistently high in each country. The death rates from cardiovascular diseases is extremely high, thereby causing about 17 million deaths every year. It is known that the onset of cardiovascular diseases is associated with vascular endothelial inflammation, and atherosclerosis is the most common of cardiovascular diseases. “Atherosclerosis” refers to a pathological state that cholesterol gradually accumulates and forms into atherosclerotic plaques on the vessel wall, this causes the vessel wall thicker, harder and losing elasticity so that blood cannot flow through the blood vessels smoothly. Atherosclerosis may cause other cardiovascular diseases, such as ischemic heart disease, stroke and peripheral arterial occlusion. It is known that patients suffering from diseases such as hypertension, hyperlipidemia and diabetes mellitus and individuals with smoking or drinking habits, sedentary lifestyles or obesity are all at high-risk of developing atherosclerosis.

Currently, hypotensive drugs, hypoglycemics drugs, hypolipidemic drugs, and anticoagulants are commonly used in clinic for controlling or treating high-risk group or patients with atherosclerosis. For patients with serious conditions, surgical operations such as endarterectomy and intravascular stent placements are adopted. However, the aforementioned surgical operations all are invasive operations, and patients generally have a slow recovery from the operations. Therefore, there is a necessity and urgency for continuously developing an effective drug or method for treating and/or alleviating cardiovascular diseases such as atherosclerosis.

It is known that an increase in the expression of ICAM-1 gene may cause vascular endothelial inflammation, thereby resulting in the onset of cardiovascular diseases. Furthermore, researches have shown that the onsets of breast cancer and adhesive capsulitis are associated with the overexpression of ICAM-1 gene, and these can be noted in “Role of ICAM1 in invasion of human breast cancer cells. Caridad Rosette et al. (2005)” and “Intercellular Adhesion Molecule-1 (ICAM-1, CD54) Is Increased in Adhesive Capsulitis. Yang-Soo Kim et al. (2013), which are entirely incorporated hereinto by reference.

Therefore, if the expression of ICAM-1 gene can be inhibited effectively, the following effects could be provided: inhibiting vascular endothelial inflammation, treating cardiovascular diseases, preventing cardiovascular diseases, treating breast cancer, preventing breast cancer, treating adhesive capsulitis, preventing adhesive capsulitis, assisting in maintenance of vascular elasticity, and assisting in protection of joint.

Inventors of the present invention discovered that euryale seed extract is effective in inhibiting the expression of ICAM-1 gene, and thus, can be used for inhibiting vascular endothelial inflammation, treating cardiovascular diseases, preventing cardiovascular diseases, treating breast cancer, preventing breast cancer, treating adhesive capsulitis, preventing adhesive capsulitis, assisting in maintenance of vascular elasticity, and/or assisting in protection of joint.

### SUMMARY OF THE INVENTION

An objective of the present invention is to provide a use of an euryale seed extract in the manufacture of a composition, wherein the composition is used for inhibiting the expression of ICAM-1 gene. Preferably, the extract is obtained by extracting an euryale seed with a polar solvent, and the polar solvent is selected from a group consisting of water, C1-C4 alcohols, and combinations thereof.

The composition provided in accordance with the present invention is a pharmaceutical composition or a food product composition, wherein the pharmaceutical composition is used for at least one of inhibiting vascular endothelial inflammation, treating cardiovascular diseases, preventing cardiovascular diseases, treating breast cancer, preventing breast cancer, treating adhesive capsulitis, and preventing adhesive capsulitis. For example, the pharmaceutical composition is used for treating and/or preventing atherosclerosis. The food product composition provided in accordance with the present invention is used for at least one of assisting in maintenance of vascular elasticity and assisting in protection of joint.

Another objective of the present invention is to provide a method for inhibiting the expression of ICAM-1 gene, comprising administering to a subject in need an effective amount of an euryale seed extract. The method of the present invention is for at least one of inhibiting vascular endothelial inflammation, treating cardiovascular diseases, preventing cardiovascular diseases, treating breast cancer, preventing breast cancer, treating adhesive capsulitis, preventing adhesive capsulitis, assisting in maintenance of vascular elasticity, and assisting in protection of joint. For example, the method of the present invention is for treating and/or preventing atherosclerosis. In the method of the present invention, the euryale seed extract can be administered to the subject in need as a form of the composition as described above.

The detailed technology and preferred embodiments implemented for the present invention are described in the following paragraphs for people skilled in this field to well appreciate the features of the claimed invention.

### BRIEF DESCRIPTION OF THE DRAWINGS

FIG. 1 shows the effect of euryale seed extract of the present invention on inhibiting the expression of ICAM-1 gene, wherein the human umbilical vein endothelial cells (HUVECs) in the control group were cultivated in a medium free of LPS and euryale seed extract for 3 hours, those in the “LPS” group were cultivated in a medium that was externally added with LPS but free of euryale seed extract for 3 hours, and those in the “extract” group were cultivated in a medium that was externally added with LPS and euryale seed extract for 3 hours (# represents the result is significantly different from that of the “LPS” group, p<0.05).

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification.

In addition, unless otherwise indicated herein, the expressions “a,” “an,” “the,” or the like recited in the specification of the present invention (especially in the claims) are intended to include both the singular and plural forms. The term “treat” or “treating” used in this specification should not be construed as treating a subject until the subject completely recovered, but should include maintaining the progression or symptoms of the diseases in a substantially static state, increasing the recovery rate of a subject, alleviating the severity of a particular condition of illness, or increasing the quality of life of a patient. The term “prevent” or “preventing” recited in this specification refers to inhibiting or avoiding a particular condition of illness from breaking out, maintaining good health in a sensitive subject, or establishing the ability of a sensitive subject to tolerate diseases. The term “regulate” or “regulating” recited in this specification refers to upregulating (includes inducing, stimulating, and enhancing) or downregulating (includes inhibiting and weakening) the physiological functions in a subject toward a normal state. The term “subject” recited in this specification refers to a mammalian, including human and non-human animals.

Euryale seed, also called as “qian shi”, is seed of a plant belonging to the family Nymphaeaceae and the genus Euryale. Inventors of the present invention discovered that the extract of euryale seed is effective in inhibiting the expression of ICAM-1 gene. Therefore, the present invention relates to uses of an euryale seed extract in inhibiting the expression of ICAM-1 gene, including the use of an euryale seed extract in the manufacture of a composition and a method of using the euryale seed extract, wherein the method comprises administering to a subject in need an effective amount of an euryale seed extract to inhibit the expression of ICAM-1 gene in the subject.

The euryale seed extract adopted in accordance with the present invention can be provided by an extraction operation. Specifically, the euryale seed extract adopted by the present invention can be provided by an operation comprising the following steps: (a) extracting an euryale seed with a solvent to provide a liquid extract; (b) centrifuging the liquid extract and then filtering the supernatant to provide a filtrate; and (c) concentrating the filtrate under vacuum to obtain an euryale seed extract.

In step (a), the extraction solvent is a polar solvent, and the polar solvent could be water, alcohols (such as C1-C4 alcohols), or combinations thereof. The amount of the solvent used in the extraction step is not critical and is generally capable of evenly dispersing the materials to be extracted. For example, in step (a), the extraction solvent and euryale seed could be used at a volume ratio ranging from 1:1 to 20:1 (extraction solvent:euryale seed). In one embodiment of the present invention, the extraction of step (a) was carried out with the use of extraction solvent and euryale seed at a volume ratio of 10:1 (extraction solvent:euryale seed).

In step (a), the extraction could be conducted for a suitable period of time depending on the extraction solvent that is adopted. When the extraction solvent and euryale seed are mixed at a volume ratio ranging from about 10:1 to 20:1 (extraction solvent:euryale seed), the extraction is usually conducted for 0.5 to 2 hours. In addition, prior to or when conducting step (a), one or more other operations such as heating, cooling, stirring, and ultrasonication could be optionally performed to further enhance the extraction efficiency. For example, in one embodiment of the present invention, a stirring operation was performed prior to conducting step (a), and then, the step (a) was conducted at 85° C. for 0.5 hours. To achieve an extraction efficiency as high as possible, the euryale seed could optionally be repeatedly extracted with the same or different extraction solvents prior to step (b), and the liquid extracts thus obtained are combined to provide the liquid extract for use in step (b). Step (b) could also be repeated.

In step (c), the temperature for conducting vacuum concentration could be optionally adjusted. For example, the vacuum concentration of step (c) could be conducted at 45° C. to 70° C. In one embodiment of the present invention, the vacuum concentration of step (c) was conducted at 55° C. to 65° C. to provide an euryale seed extract as needed.

The composition provided in accordance with the present invention is a pharmaceutical composition or a food product composition, wherein the pharmaceutical composition could be used for at least one of inhibiting vascular endothelial inflammation, treating cardiovascular diseases, preventing cardiovascular diseases, treating breast cancer, preventing breast cancer, treating adhesive capsulitis, and preventing adhesive capsulitis. Particularly, the cardiovascular disease is atherosclerosis. The food product composition provided in accordance with the present invention could be used for at least one of assisting in maintenance of vascular elasticity and assisting in protection of joint.

Depending on the desired purpose, the pharmaceutical composition in accordance with the present invention could be provided in any suitable form without specific limitations. For example, the pharmaceutical composition could be administered to a subject in need by an oral or parenteral (such as transdermal, intravenous or subcutaneous) route, but is not limited thereby. Depending on the form and purpose, suitable carriers can be chosen and used to provide the pharmaceutical composition. Examples of the carriers include excipients, diluents, auxiliaries, stabilizers, absorbent retarders, disintegrating agent, hydrotropic agents, emulsifiers, antioxidants, adhesives, binders, tackifiers, dispersants, suspending agents, lubricants, hygroscopic agents, etc.

As a dosage form for oral administration, the pharmaceutical composition could comprise any pharmaceutically acceptable carriers that will not adversely affect the desired effects of the active ingredient (i.e., euryale seed extract). Examples of suitable carriers include, but is not limited to, water, saline, dextrose, glycerol, ethanol or its analogs, cellulose, starch, sugar bentonite, and combinations thereof. The pharmaceutical composition could be provided in any suitable form for oral administration, such as in a form of a tablet (e.g., sugar-coated tablet), a pill, a capsule, granules, a pulvis, a fluidextract, a solution, syrup, a suspension, a tincture, etc.

As a dosage form for transdermal administration, the pharmaceutical composition could be provided in a form such as a patch, an emulsion, a cream, a gel (such as a hydrogel), a paste (such as a dispersing paste, an ointment), a spray, or a solution (such as a suspension) for external use, but is not limited thereby.

As for the form of injections or drips suitable for subcutaneous administration or intravenous administration, the pharmaceutical composition could comprise one or more ingredient(s), such as an isotonic solution, a salt-buffered saline (e.g., phosphate-buffered saline or citrate-buffered saline), a hydrotropic agent, an emulsifier, a 5% sugar solution, and other carriers to provide the pharmaceutical composition as an intravenous infusion, an emulsified intravenous infusion, a powder for injection, a suspension for injection, or a powder suspension for injection, etc. Alternatively, the pharmaceutical composition could be prepared as a pre-injection solid. The pre-injection solid could be provided in a form which is soluble in other solutions or suspensions, or in an emulsifiable form. A desired injection is provided by dissolving the pre-injection solid in other solutions or suspensions or emulsifying it prior to being administered to a subject in need.

Depending on the need, age, body weight and health conditions of the subject, the pharmaceutical composition provided in accordance with the present invention could be dosed at various administration frequencies, such as once a day, multiple times a day, once every few days, etc. The ratio of amount of euryale seed extract in the pharmaceutical composition provided in accordance with the present invention could be adjusted depending on the requirements of practical application. In addition, the pharmaceutical composition could optionally further comprise one or more other active ingredient(s) (such as hypolipidemic drugs, hypotensive drugs, and anticoagulants, etc.), or to be used in combination with a medicament comprising one or more other active ingredient(s), to further enhance the effects of the pharmaceutical composition, or to increase the application flexibility and adaptability of the preparation thus provided, as long as the other active ingredients will not adversely affect the desired effects of the active ingredient of the present invention (i.e., euryale seed extract).

Optionally, the pharmaceutical composition or food product composition provided in accordance with the present invention could further comprise a suitable amount of additives, such as a flavoring agent, a toner, or a coloring agent for enhancing the palatability and the visual perception of the pharmaceutical composition or food product composition, and/or a buffer, a conservative, a preservative, an antibacterial agent, or an antifungal agent for improving the stability and storability of the pharmaceutical composition or food product composition.

The food product composition provided in accordance with the present invention could be a health food, a dietary supplement, a functional food, a nutritional supplement food or a special nutritional food, and could be manufactured as a milk product, a processed meat product, breadstuff, pasta, cookies, troche, capsules, a fruit juice, a tea product, a sport beverage, a nutritional beverage, etc., but is not limited thereby. Preferably, the food product composition provided in accordance with the present invention is a health food.

Depending on the age, body weight and health conditions of the subject, the health food, a dietary supplement, a functional food, a nutritional supplement food and a special nutritional food provided in accordance with the present invention could be taken at various frequencies, such as once a day, multiple times a day, once every few days, etc. The amount of the euryale seed extract in the health food, dietary supplement, functional food, nutritional supplement food or special nutritional food provided in accordance with the present invention could also be adjusted, preferably to the amount that should be taken daily, depending on the specific population.

The recommended dosage, use standards and use conditions for a specific population (e.g., stroke patients, heart disease patients, and pregnant women, etc), or recommendations for use in combination with other food products or pharmaceuticals could be labeled on the exterior package of the health food, dietary supplement, functional food, nutritional supplement food and/or special nutritional food provided in accordance with the present invention. Thus, it is suitable for the user to take the health food, dietary supplement, functional food, nutritional supplement food and/or special nutritional food by him- or herself safely and securely without the instructions of a doctor, pharmacist, or related executive. In the food product composition provided in accordance with the present invention, the applied type and uses in related application of the euryale seed extract are all in line with the above descriptions.

The present invention also provides a method for inhibiting the expression of ICAM-1 gene, comprising administering to a subject in need an effective amount of an euryale seed extract. Preferably, the method of the present invention is for treating, preventing, or regulating diseases or physiological functions associated with ICAM-1 gene. Particularly, the method of the present invention is for treating and/or preventing cardiovascular diseases. “A subject in need” in the aforementioned description refers to a subject suffering from the diseases associated with the mutation or overexpression of ICAM-1 gene. The euryale seed extract adopted in accordance with the method of the present invention could be administered to the subject in need as the form of a composition. The applied type, applied route, applied form, applied frequency and uses in related application of the composition are all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

The sources of materials used in the examples are listed as follows:
1. Euryale seed: purchased from Season Organic International Co., Ltd (Taiwan).
2. Human umbilical vein endothelial cell (HUVEC): purchased from BCRC, product number: H-UV001.
3. EC medium: M200 medium (purchased from Gibco, product number: M-200-500)+10% LSGS (Low Serum Growth Supplement; purchased from Gibco, product number: S-003-10).
4. LPS (Lipopolysaccharides): purchased from Gibco, product number: 00-4976.
5. RNA extraction kit: purchased from GENEmark.
6. SuperScript® III Reverse Transcriptase: purchased from Invitrogen.
7. KAPA SYBR FAST qPCR kit: purchased from KAPA Biosystems.
8. Step One Plus system: purchased from ABI.

### Preparation Example

### Preparation of Euryale Seed Extract

Euryale seed was subjected to the following operation comprising the following steps, to provide an euryale seed extract:
1. Mixing euryale seed (without peeling) with water at a volume ratio of 1:10 (euryale seed:water) to provide a mixture, and then subjecting the mixture to an extraction at 85° C. for 0.5 hours to provide a liquid extract;
2. Centrifuging the liquid extract obtained from step 1, and then filtering the supernatant with a mesh filter to provide a filtrate; and
3. Concentrating the filtrate obtained from step 2 under vacuum at 55° C. to 65° C. to provide an euryale seed extract.

### Example 1: Effect of Euryale Seed Extract on Inhibiting the Expression of ICAM-1 Gene

Human umbilical vein endothelial cells seeded in a 6-well plate (1×10⁵ cells/well) were cultivated for 1 hour. Then, the cells were divided into the control group, “LPS” group and “Extract” group and independently cultivated with the following media for 3 hours:
1. Control group: EC medium;
2. “LPS” group: EC medium that was externally added with LPS (to the final concentration of 1 μg/ml); and
3. “Extract” group: EC medium that was externally added with LPS (to the final concentration of 1 μg/ml) and euryale seed extract (to the final concentration of 0.5 mg/ml) obtained from [Preparation Example].

Thereafter, cells of each group were harvested and subjected to an RNA extraction with an RNA extraction kit. The RNA was reverse transcribed into cDNA with a reverse transcriptase. Then, the cDNA was subjected to a quantitative polymerase chain reaction (qPCR) by an ABI Step One Plus system and a KAPA SYBR FAST qPCR kit, to determine the expression level of ICAM-1 gene in the cells of each group.

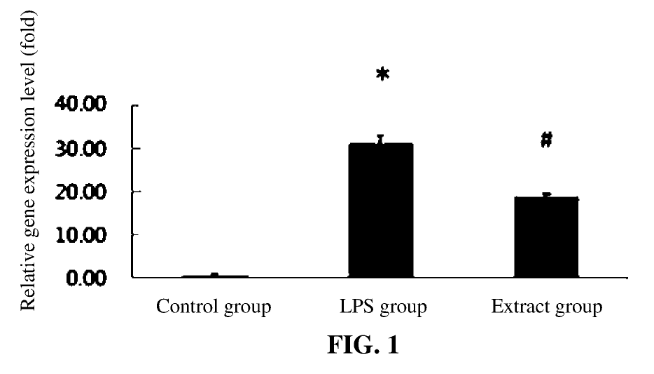

The above experimentation was repeated three times, and the results obtained from the three experimentations were averaged. Then, the result of control group was used as a basis (i.e., the gene expression level of control group was set as 1-fold) to calculate the relative gene expression levels of the “LPS” group and “Extract” group. The results are shown in FIG. 1.

As shown in FIG. 1, in comparison with that of control group, the expression level of ICAM-1 gene of the “LPS” group significantly increased. This result indicates that LPS can indeed induce the vascular endothelial inflammation. In comparison with that of “LPS” group, the expression level of ICAM-1 gene of the “Extract” group significantly decreased. This result indicates that the euryale seed extract of the present invention can significantly reduce the LPS-induced increment in the expression level of ICAM-1 gene. The aforementioned results indicate that the euryale seed extract of the present invention can indeed effectively inhibit the expression level of ICAM-1 gene, and thus, can be used for inhibiting vascular endothelial inflammation, treating cardiovascular diseases, preventing cardiovascular diseases, treating breast cancer, preventing breast cancer, treating adhesive capsulitis, preventing adhesive capsulitis, assisting in maintenance of vascular elasticity, and assisting in protection of joint.

What is claimed is:

1. A method for inhibiting expression of ICAM-1 gene, comprising administering to a subject in need an effective amount of an euryale seed extract.

2. The method as claimed in claim 1, wherein the extract is obtained by extracting an euryale seed with a polar solvent, and the polar solvent is selected from a group consisting of water, C1-C4 alcohols, and combinations thereof.

3. The method as claimed in claim 1, which is for inhibiting vascular endothelial inflammation, and the extract is administered to the subject as a pharmaceutical composition.

4. The method as claimed in claim 1, which is for treating cardiovascular diseases, preventing cardiovascular diseases, treating breast cancer, preventing breast cancer, treating adhesive capsulitis, and/or preventing adhesive capsulitis, and the extract is administered to the subject as a pharmaceutical composition.

5. The method as claimed in claim 4, wherein the cardiovascular disease is atherosclerosis.

6. The method as claimed in claim 1, which is for assisting in maintenance of vascular elasticity, and/or assisting in protection of joint, and the extract is administered to the subject as a food product composition.

7. The method as claimed in claim 2, which is for inhibiting vascular endothelial inflammation, and the extract is administered to the subject as a pharmaceutical composition.

8. The method as claimed in claim 2, which is for treating cardiovascular diseases, preventing cardiovascular diseases, treating breast cancer, preventing breast cancer, treating adhesive capsulitis, and/or preventing adhesive capsulitis, and the extract is administered to the subject as a pharmaceutical composition.

9. The method as claimed in claim 8, wherein the cardiovascular disease is atherosclerosis.

10. The method as claimed in claim 2, which is for assisting in maintenance of vascular elasticity, and/or assisting in protection of joint, and the extract is administered to the subject as a food product composition.
